(19) United States
(12) Patent Application Publication
(10) Pub. No.: US 2022/0378545 A1
(43) Pub. Date: Dec. 1, 2022

(54) ARTICULATED ARM FOR HOLDING AN ELONGATED FLEXIBLE MEDICAL INSTRUMENT

(71) Applicant: ROBOCATH, Rouen (FR)

(72) Inventors: Marc BOULANGE, Gruchet-Saint-Simeon (FR); Philippe BENCTEUX, Saint Martin Du Vivier (FR)

(21) Appl. No.: 17/776,512

(22) PCT Filed: Nov. 4, 2020
(86) PCT No.: PCT/FR2020/051991

§ 371 Date: May 12, 2022

(30) Foreign Application Priority Data
Nov. 15, 2019 (FR) FR1912806

(51) Int. Cl. A61B 90/50; F16M 11/06; F16D 55/226

(52) U.S. Cl. CPC A61B 90/50; F16M 11/06; F16D 55/226; A61B 2090/508; A61B 2017/00539

(57) ABSTRACT

Disclosed is an articulated support arm for an elongated flexible medical instrument, including: a segment rotating about an axis of rotation, a radial brake for preventing rotation of the segment about the axis of rotation, including: a supply of hydraulic fluid, a piston whose stroke along the direction of the axis of rotation, due to the pressure of the hydraulic fluid, prevents rotation of the segment about the axis of rotation, the piston being an annular piston having a hollow center about the axis of rotation, the supply of hydraulic fluid passing through the hollow center.

### CROSS-REFERENCE RELATED TO PRIORITY APPLICATIONS

This application is the U.S. national phase of International Application No. PCT/FR2020/051991 filed Nov. 4, 2020 which designated the U.S. and claims priority to FR1912806 filed Nov. 15, 2019, the entire contents of each of which are hereby incorporated by reference.

### BACKGROUND OF THE INVENTION

### Field of the Invention

The invention relates to an articulated support arm for an elongated flexible medical instrument, incorporating one or more segments rotating about one or more axes of rotation.

### Description of the Related Art

In prior art concerning an articulated arm carrying a catheter and comprising one or more segments rotating about an axis of rotation, it is known to have constant friction between the segment and the axis of rotation around which the segment rotates.

A first disadvantage of this prior art is that the rotation of the segment about its axis of rotation is quite stiff, in other words is not very smooth. This first disadvantage is a rotation that is not sufficiently smooth.

A second disadvantage of this prior art is that, when the segment remains stationary around its axis of rotation, in fact it is not truly prevented from rotating and a noticeable bump that is not very significant, for example caused by medical staff or by the patient, can move it about its axis of rotation, thus losing the correct angular position in which this segment was rotationally fixed. This second disadvantage is a stationary position that is not sufficiently prevented from rotating about the axis of rotation.

A third disadvantage is the wear over time of this constant friction, the stationary rotational position of the segment being less and less properly maintained as time passes and as the articulated arm is used.

### SUMMARY OF THE INVENTION

The aim of the invention is to improve at least partially one or more of the above disadvantages, while being of limited overall size.

The invention proposes improving the compromise between rotational fluidity and rotation-prevention efficiency, on the one hand by having a prevention brake which offers a more fluid rotation of the segment of the articulated arm when in the disengaged position than in this prior art, and on the other hand by having a prevention brake which offers, in the prevention position, a stationary position of the articulated arm segment that is better prevented from rotating than in this prior art, in addition with a prevention brake that undergoes less wear over time, all of this while having a prevention brake of relatively small footprint and a prevention brake whose hydraulic fluid supply is also of relatively small footprint.

A supply of hydraulic fluid that pushes a piston and travels from one side or the other of the piston it pushes could be provided. However, in this case, the invention takes into consideration the longer hydraulic fluid supply pipes extending around the piston and rotating segment that would be required, and these lengthened hydraulic fluid supply pipes could then interfere with the rotational movements of either the rotating segment or of one of the rotating segments in the articulated arm. In addition, these supply pipes would take up space. The ease of use would be reduced and the size would be increased accordingly.

According to the invention, for a general improvement in the compromise between all the numerous problems existing in the prior art or considered by the invention, an articulated arm which improves this compromise is provided.

To this end, the invention proposes an articulated support arm for an elongated flexible medical instrument, comprising: a segment rotating about an axis of rotation, a radial brake for preventing rotation of the segment about the axis of rotation, comprising: a hydraulic fluid supply, a piston whose stroke along the direction of the axis of rotation, due to the pressure of the hydraulic fluid, prevents rotation of the segment about the axis of rotation, the piston being an annular piston having a hollow center about the axis of rotation, the hydraulic fluid supply passing through said hollow center.

To this end, the invention also provides a method for preventing the mobility of an articulated support arm for an elongated flexible medical instrument, comprising a segment that rotates about an axis of rotation and a radial brake for preventing rotation of the segment about the axis of rotation, comprising: a supplying of hydraulic fluid through a hollow center of an annular piston of the radial brake, a stroke of said piston along the direction of the axis of rotation, due to the pressure of said hydraulic fluid, so as to prevent rotation of the segment about the axis of rotation.

According to preferred embodiments, the invention comprises one or more of the following features which may be used separately or in a combination of some or all of these features, and with one of the above objects of the invention.

Preferably, said stroke of said piston prevents the rotation of said segment about the axis of rotation, but does not lock it.

Thus, in an emergency for example, the rotating segment is only prevented from rotating but is not locked, and therefore it can be released more quickly and easily by the user, for example by pressing an emergency button which will command the emergency release of the segment(s) in rotation and even possibly of the entire articulated arm.

As an indication, we can distinguish between:
Braking without rotation prevention:
   If the arm is pushed slightly by an inadvertent bump, the arm moves and is pushed out of position, but less so than if there was no braking;
   Rotation prevention without locking:
   If the arm is pushed slightly by an inadvertent bump, the arm does not move but remains in position,
   In order for the arm to move and be pushed out of position, it must either be struck with a certain amount of force, or the pressure of the hydraulic fluid must be released;
   Rotation prevention with locking:
   In order to move and push the arm out of position, some or all of the rotation-prevention and locking mechanism must be broken (or damaged), unless of course it is unlocked first.

Preferably, the articulated arm also comprises: an axial pivot which physically defines the axis of rotation and around which the segment rotates, the axial pivot passing through said hollow center, a central channel for the hydraulic fluid supply, passing inside the axial pivot and through said hollow center.

Thus, the size of the rotation-preventing brake and of the hydraulic fluid supply is even further reduced.

Indeed, the additional problem considered by the invention, of reducing the total length of the hydraulic fluid supply pipes to the piston, is then optimal because this total length of the hydraulic fluid supply pipes is then reduced as much as possible. In addition, dissymmetries of the rotating segment about the axis of rotation are then also reduced as much as possible, thus minimizing the risk of an imbalance or lack of equilibrium of the rotating segment around its axis of rotation, not to mention the risk that part of the human body of either the patient or the user of the articulated arm could catch on the hydraulic fluid supply pipes.

Preferably, the central channel for the hydraulic fluid supply extends in the direction of the axis of rotation.

The footprint of the rotation-preventing brake and of the hydraulic fluid supply is thus further reduced.

Preferably, the central channel for the hydraulic fluid supply extends along the axis of rotation. The footprint of the rotation-preventing brake and of the hydraulic fluid supply is thus further reduced. In addition, the overall inertia of the axial pivot is generally better balanced around the axis of rotation.

Preferably, the articulated arm also comprises a radial channel for the hydraulic fluid supply, extending radially to the axis of rotation and connecting the central channel to the annular piston.

The footprint of the rotation-preventing brake and of the hydraulic fluid supply is thus further reduced.

Preferably, at least one of the axial ends of the axial pivot comprises a hydraulic connection including a rotating gasket.

The amplitude of the movements of the articulated arm is thus generally increased, while maintaining a relatively small footprint of the rotation-preventing brake and of the hydraulic fluid supply.

Preferably, the segment rotates about the axial pivot by means of a needle bearing or a plurality of needle bearings which is/are radially positioned between the axial pivot and the segment.

The rotation of the segment about its axis of rotation is thus better balanced and the articulated arm experiences less overall wear over time. In addition, the footprint of the rotating segment, and therefore also the overall footprint of the articulated arm, is further reduced slightly. Preferably, the articulated arm also comprises two of said rotation-preventing radial brakes with annular piston, located respectively at two axial ends of the axial pivot.

The rotation of the segment around its axis of rotation, as well as the prevention of its rotation, are thus better balanced and distributed, indirectly leading to a reduction in footprint and less wear over time.

Preferably, the segment has two ends which respectively rotate about two of said axial pivots whose axes of rotation have the same direction.

The degrees of freedom in positioning the articulated arm are thus generally improved and enriched.

Preferably, the segment is rectilinear and inclined relative to the direction of the axes of rotation of the two axial pivots. Advantageously, the inclination of the rectilinear segment relative to the direction of the axes of rotation of the two axial pivots is between 15° (15 degrees) and 75° (75 degrees), more advantageously between 30° and 75°, even more advantageously 45° and 75°, or even between 60° and 75°.

The overall footprint of the segment, rotation-preventing brakes, and hydraulic fluid supply is thus reduced.

Preferably, the two axial pivots are interconnected by a hydraulic fluid supply line.

The hydraulic fluid supply thus provides a path that is simpler and shorter overall.

Preferably, the hydraulic fluid supply line is rectilinear and orthogonal to the direction of the axes of rotation of the two axial pivots.

The hydraulic fluid supply thus provides a path that is shorter and occupies less space overall. Preferably, the radial brake comprises: a lining attached to the annular piston, a lining attached to the segment, an unattached lining which is not attached to either the annular piston or the segment, the material of the unattached lining being less hard than the material or materials of the attached linings, the attached linings and the unattached lining being arranged relative to one another such that, when the hydraulic fluid pushes the annular piston, the unattached lining is clamped between the two attached linings so as to prevent rotation of the segment about the axis of rotation, advantageously without locking it.

The trade-off between structural simplicity and operating efficiency of the rotation-preventing brake is thus improved overall.

Preferably, the lining attached to the annular piston is made of steel, the lining attached to the segment is made of steel, and the unattached lining is made of plastic.

Thus, the trade-off between structural simplicity and operating efficiency of the rotation-preventing brake is generally improved. In addition, only the unattached lining undergoes wears and may need to be changed later on, which makes the articulated arm easier to service and maintain.

The articulated arm is the articulated arm of a surgical robot carrying an elongated flexible medical member, for example of the type including a catheter and a catheter guide and a guide catheter. This surgical robot is used to introduce the elongated flexible medical member into the circulatory system of a patient, for example.

Preferably, the articulated arm comprises at least three segments articulated to rotate with respect to each other about a same rotation direction, which include: a proximal segment located closest to the operating table when the articulated arm is fixed to this operating table, a distal segment carrying the elongated flexible medical instrument, and an intermediate segment located between the proximal segment and the distal segment.

Preferably, the articulated arm comprises at least four segments articulated to rotate with respect to each other about a same rotation direction, which include: a proximal segment located closest to the operating table when the articulated arm is fixed to this operating table, a distal segment carrying the elongated flexible medical instrument, and at least two intermediate segments located between the proximal segment and the distal segment.

Thus, the rotational fluidity (when the segment(s) are in motion), the rotation-prevention efficiency (when the segment(s) are stationary), as well as the overall footprint of the articulated arm, are all the greater when the structure of the articulated arm is complex and long.

Preferably, the elongated flexible medical instrument comprises a catheter and/or a catheter guide and/or a guide catheter.

Other features and advantages of the invention will become apparent from reading the following description of a preferred embodiment of the invention, given as an example and with reference to the accompanying drawings.

### BRIEF DESCRIPTION OF DRAWINGS

FIG. 1 shows an example of an articulated arm portion comprising several rotating segments according to one embodiment of the invention.

FIG. 2 shows an example of a rotating segment portion of an articulated arm according to one embodiment of the invention. FIG. 2 is a detailed view of part of FIG. 1.

FIG. 3 schematically represents an example of the operation of a segment-rotation-preventing radial brake in the resting position of the rotation-preventing radial brake, but without necessarily abiding by the structure and proportions of the articulated arm according to an embodiment of the invention.

FIG. 4 schematically represents an example of the operation of a segment-rotation-preventing radial brake in the applied-pressure position of the rotation-preventing radial brake, but without necessarily abiding by the structure and proportions of the articulated arm according to an embodiment of the invention.

### DESCRIPTION OF THE PREFERRED EMBODIMENTS

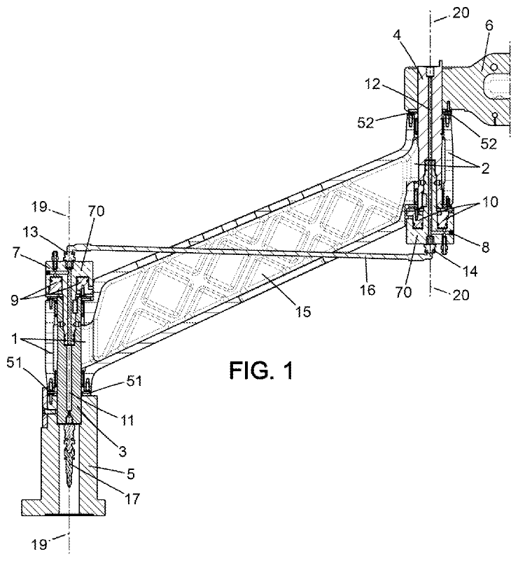

In all of FIGS. 1-4, the elongated flexible medical instrument comprises a catheter and/or a catheter guide and/or a guide catheter.

FIG. 1 shows an example of an articulated arm portion comprising several rotating segments according to one embodiment of the invention.

A segment 15 of an articulated arm has a first portion 1 rotating about a first axis of rotation 19 located at the center of a first axial pivot 3, and has a second portion 2 rotating about a second axis of rotation 20 located at the center of a second axial pivot 4.

The first axial pivot 3 is integral with another segment 5; it is surmounted, at one of its ends, by a rotation-preventing radial brake 7 itself surmounted by a hydraulic connection 13 extended by a hydraulic fluid supply line 16. The center of the first axial pivot 3 is hollow at the first axis of rotation 19, comprising at its center a channel 11 in which the hydraulic fluid circulates. The hydraulic fluid arriving through the channel 11 is brought to the rotation-preventing radial brake 7 and more specifically to the annular piston 9 itself in order to exert hydraulic thrust thereon which will prevent the relative pivoting about the first axis of rotation 19 of segment 15 with respect to the first axial pivot 3 and with respect to segment 5 which is integral with the first axial pivot 3. The first axial pivot 3 is extended, at the other of its ends, by a hydraulic fluid supply inlet 17. Near this other end, the first axial pivot 3 comprises a complementary rotation-preventing radial brake 51 which transmits the hydraulic pressure from rotation-preventing radial brake 7. The joint presence of rotation-preventing radial brake 7 and complementary rotation-preventing radial brake 51, respectively located on both sides towards the two longitudinal ends of the first axial pivot 3, allows better distribution and better balancing of the hydraulic braking pressure along the first axial pivot 3.

The second axial pivot 4 is integral with another segment 6; it is surmounted, at one of its ends, by a rotation-preventing radial brake 8 itself surmounted by a hydraulic connection 14 arranged in the extension of the hydraulic fluid supply line 16. The center of the second axial pivot 4 is hollow at the second axis of rotation 20, comprising at its center a channel 12 in which the hydraulic fluid circulates. The hydraulic fluid arriving through the channel 12 is brought to the rotation-preventing radial brake 10 and more specifically to the annular piston 10 in order to exert hydraulic thrust thereon which will prevent the relative pivoting about the second axis of rotation 20 of segment 15 with respect to the second axial pivot 4 and with respect to segment 6 which is integral with the first axial pivot 4. The second axial pivot 4 comprises, near the other of its ends, a complementary rotation-preventing radial brake 52 which transmits the hydraulic pressure from rotation-preventing radial brake 8. The joint presence of rotation-preventing radial brake 8 and complementary rotation-preventing radial brake 52, respectively located on both sides towards the two longitudinal ends of the second axial pivot 4, allows better distribution and better balancing of the hydraulic braking pressure along the second axial pivot 4.

The first axis of rotation 19 and the second axis of rotation 20 are parallel to one another, and are advantageously both vertical in FIG. 1. The first axis of rotation 19 and the second axis of rotation 20 respectively correspond to the axes of symmetry of the first axial pivot 3 and second axial pivot 4, as well as to the axes of symmetry of the first channel 11 and second channel 12. When the hydraulic fluid, which is preferably a liquid, but could also be a pressurized gas, exerts hydraulic thrust on rotation-preventing radial brake 7, the relative position between segment 15 and segment 5 remains immobilized in rotation. When this hydraulic fluid does not or no longer exerts this hydraulic thrust on rotation-preventing radial brake 7, the relative position between segment 15 and segment 5 is or becomes free to rotate. The hydraulic thrust or hydraulic pressure exerted by rotation-preventing radial brake 7 is transmitted to complementary rotation-preventing radial brake 51. Piston 9, of which the thrust along the direction of the first axis of rotation 19, due to the pressure of the hydraulic fluid, prevents the relative rotation of segment 15 with respect to segment 5 about the first axis of rotation 19, is an annular piston 9 having a hollow center 70 around the first axis of rotation 19, the hydraulic fluid supply traveling through channel 11 forming a hollow center in the first axial pivot 3. When the hydraulic fluid exerts hydraulic thrust on rotation-preventing radial brake 8, the relative position between segment 15 and segment 6 remains immobilized in rotation. When this hydraulic fluid does not or no longer exerts this hydraulic thrust on rotation-preventing radial brake 8, the relative position between segment 15 and segment 6 is or becomes free to rotate. The hydraulic thrust or hydraulic pressure exerted by rotation-preventing radial brake 8 is transmitted to complementary rotation-preventing radial brake 52.

Piston 10, of which the stroke along the direction of the second axis of rotation 20, due to the pressure of the hydraulic fluid, prevents the relative rotation of segment 15 with respect to segment 6 about the second axis of rotation 20, is an annular piston 10 having a hollow center 70 around the second axis of rotation 20, the hydraulic fluid supply traveling through the channel 12 forming a hollow center in the second axial pivot 4.

As an alternative to complementary rotation-preventing radial brake 51 which only transmits the hydraulic thrust of rotation-preventing radial brake 7, another rotation-blocking radial brake with an annular piston may be used for segment 15 to prevent its rotation relative to segment 5 about axis of rotation 19, the two rotation-preventing radial brakes then being respectively located at two axial ends of the first axial pivot 3, and both exerting a hydraulic thrust to prevent the relative rotation of segment 15 and segment 5. The same replacement of complementary rotation-preventing radial brake 52 by another rotation-preventing radial brake itself exerting a hydraulic thrust similar to rotation-preventing radial brake 8 is also conceivable.

The hydraulic fluid comes from the hydraulic fluid supply inlet 17 feeding through segment 5:
which traverses channel 11 in the first axial pivot 3,
   then is sent into rotation-preventing radial brake 7 and even all the way to annular piston 9,
   and simultaneously is sent into hydraulic connection 13,
   from where it circulates through the supply line 16 to reach hydraulic connection 14,
   then is sent into rotation-preventing radial brake 8 and even all the way to annular piston 10,
   and simultaneously is sent into channel 12 of the second axial pivot 4 if there is a second rotation-preventing radial brake exerting a hydraulic thrust,
   or stopping at rotation-preventing radial brake 8 and annular piston 10 if there is only one complementary rotation-preventing radial brake 52,
   or traveling on to another supply line if there is yet another rotation to be prevented between segment 6 and another segment more distal than segment 6.

The two axial pivots 3 and 4 are interconnected by the hydraulic fluid supply line 16 which is rectilinear and orthogonal to the direction of the axes of rotation 19 and 20 (same direction for these two axes of rotation 19 and 20) of the two axial pivots 3 and 4.

In one embodiment, the articulated arm comprises three articulated segments that rotate with respect to one another about the same rotation direction. Segment 5 is, for example, a proximal segment, in other words located closest to the operating table when the articulated arm is fixed to this operating table. Segment 6 is, for example, a distal segment carrying the elongated flexible medical instrument. Segment 15 is, for example, an intermediate segment located between proximal segment 5 and distal segment 6.

In another embodiment, the articulated arm comprises four or five articulated segments that rotate with respect to one another about the same rotation direction. Segment 5 is, for example, a proximal segment, in other words located closest to the operating table when the articulated arm is fixed to this operating table, or else is an intermediate segment connected to the proximal segment. Segment 6 is, for example, a distal segment carrying the elongated flexible medical instrument, or else is an intermediate segment connected to the distal segment. Segment 15 is, for example, an intermediate segment located between proximal segment 5 and distal segment 6, and connected by one of its ends to either proximal segment 5 or intermediate segment 5, depending on the number of segments comprised in the articulated arm, while also being connected by the other of its ends to either distal segment 6 or intermediate segment 6, depending on the number of segments comprised in the articulated arm.

The stroke of pistons 9 and 10 respectively prevents, but does not lock, the rotation of segment 15 with respect to segments 5 and 6 respectively rotating about the first axis of rotation 19 and about the second axis of rotation 20.

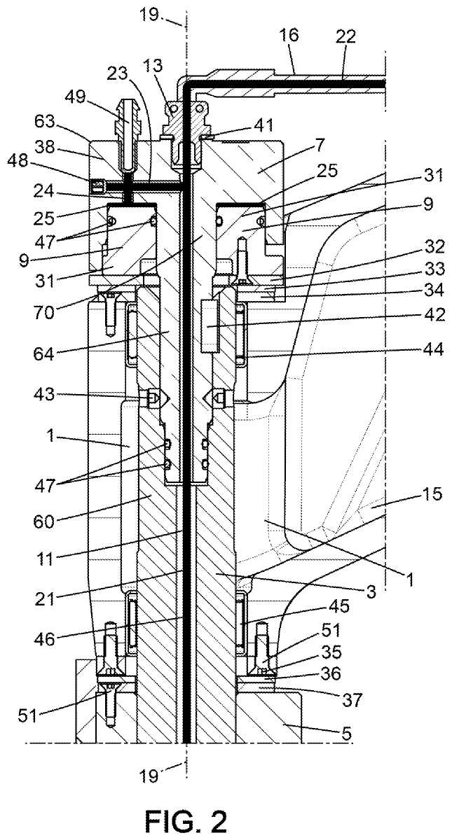

FIG. 2 shows an example of a rotating segment portion of an articulated arm according to one embodiment of the invention. FIG. 2 is a detailed view of part of FIG. 1. FIG. 2 shows the area located around the first axial pivot 3 at one of the ends of segment 15 (portion 1) which rotates about this first axial pivot 3 and of segment 5 which is integral with this first axial pivot 3. The structure and operation at the area located around the second axial pivot 4 at the other end of segment 15 which rotates about this second axial pivot 4, and of segment 6 which is integral with this second axial pivot 4, are similar.

The first axial pivot 3 comprises a main body 60 in which is fitted a radial brake 7 body 38 itself comprising a head 63, including an annular cavity filled by annular piston 9, and an axial protuberance 64 passing through annular piston 9 which is hollow at its center. The axial protuberance 64 is itself hollow at its center.

Channel 11 comprises the central channel 21 passing successively through the hollow center of the first axial pivot 3 then the hollow center of the head 63 of radial brake 7 and then hydraulic connection 13 before branching at a right angle into the supply line 16. Hydraulic connection 13 comprises a rotating gasket to ensure the relative rotation, about the first axis of rotation 19, between the supply line 16 and the head 63 of radial brake 7. The connection located between hydraulic connection 13 and the head 63 of radial brake 7 is made fluid-tight by a sealing washer 41, preferably of copper.

Channel 11 comprises a bypass in the head 63 which includes a radial channel 23 then an elbow 24 and then a ring 25 which fills with hydraulic fluid 46 that presses on the upper surface of the body 31 of annular piston 9 and exerts hydraulic thrust on annular piston 9. A hydraulic fluid 46 circulates in channel 11, preferably liquid.

The body 31 of annular piston 9 transmits this hydraulic pressure to a stack of annular linings, successively to an annular lining 32 preferably made of steel and attached to the body 31 of annular piston 9, then to an unattached annular lining 33 preferably made of plastic, then to a lining 34 preferably made of steel and attached to portion 1 of segment 15. Next, segment 15 transmits this hydraulic pressure to a stack of annular linings, successively to an annular lining 35 preferably made of steel and attached to portion 1 of segment 15, then to an annular lining 36 preferably made of plastic and unattached, then to a lining 37 preferably made of steel and attached to the first axial pivot 3 and/or to segment 5 which is integral with the first axial pivot 3. The various attached and unattached linings are arranged relative to each other so that, when the hydraulic fluid 46 pushes annular piston 9, unattached lining 33 is clamped between the two attached linings 32 and 34 so as to prevent rotation of the segment about the axis of rotation, advantageously without locking it, and also so that unattached lining 36 is clamped between the two attached linings 35 and 37 so as to prevent rotation of segment 15 about axis of rotation 19, advantageously without locking it.

Segment 15 rotates about the first axial pivot 3 by means of a needle bearing 44, or even (in FIG. 2) several needle bearings 44 and 45 located radially between the body 60 of the first axial pivot 3 and the first portion 1 of segment 15.

The axial protuberance 64 of the body 38 of radial brake 7 is rotationally locked about the first axis of rotation 19 by being secured to the body 60 of the first axial pivot 3 by means of a pin 42. The axial protuberance 64 of the body 38 of radial brake 7 is translationally locked along the first axis of rotation 19 by being secured to the body 60 of the first axial pivot 3 by means of several retaining screws 49, at least two, regularly distributed around the first axis of rotation 19, in other words also around the body 60 of the first axial pivot 3, again in other words around the axial protuberance 64 of radial brake 7. O-rings 47 ensure fluidtightness, on the one hand between the axial protuberance 64 of radial brake 7 and the inside of the body 60 of the first axial pivot 3, and on the other hand between annular piston 9 and the hollow annular chamber 65 of the head 63 of radial brake 7, said annular chamber 65 being where annular piston 9 slides in translation along the first axis of rotation 19 when the hydraulic fluid 46 enters the ring 25 and begins to push annular piston 9 downwards.

One end, which opens to outside the head 63 of radial brake 7, of the radial channel 23 is closed by a plug 48. The extension of the elbow 24 through the radial channel 23 is closed by a bleeder screw 49.

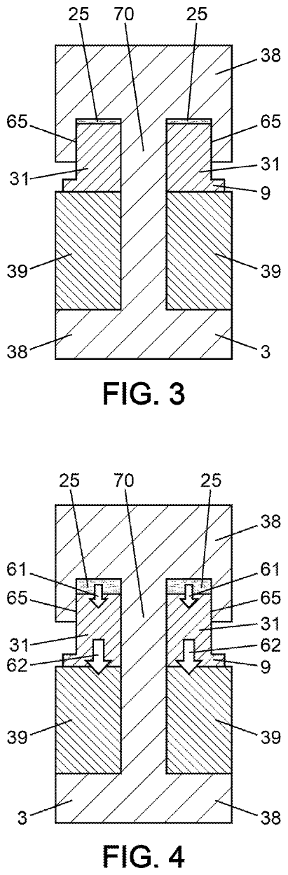

FIG. 3 schematically represents an example of the operation of a segment-rotation-preventing radial brake in the resting position of the rotation-preventing radial brake, but without necessarily abiding by the structure and proportions of the articulated arm according to an embodiment of the invention.

The operation of the segment-rotation-preventing radial brake 7 in the resting position of the rotation-preventing radial brake 7 is as follows:
the ring 25 of the annular chamber 65 of the body 38 of the first axial pivot 3, slightly filled or not filled with hydraulic fluid, is thin and therefore only slightly presses on the body 31 of annular piston 9,
   said body 31 of annular piston 9 exerts little or no compression on the movable body 39, this movable body 39 being formed by the stack of linings described in FIG. 2 and by the portion 1 of segment 15,
   in return, said movable body 39 exerts little or no pressure on the lower part of the body 38 of radial brake 7,
   such that the movable body 39 can rotate freely around the body 38 traversing it.

FIG. 4 schematically represents an example of the operation of a segment-rotation-preventing radial brake in the applied-pressure position of the rotation-preventing radial brake, but without necessarily abiding by the structure and proportions of the articulated arm according to an embodiment of the invention.

The operation of segment-rotation-preventing radial brake 7 in the applied-pressure position of rotation-preventing radial brake 7 is as follows:
the ring 25 of the annular chamber 65 of the body 38 of the first axial pivot 3, filled with hydraulic fluid, becomes increasingly thick (the thickness being the dimension parallel to the direction of the axis of rotation) as it fills with hydraulic fluid, and therefore presses more and more on the body 31 of annular piston 9 which slides downward, parallel to the axis of rotation of the segment around the axial pivot, this pressure or hydraulic thrust being symbolized by arrow 61,
   said body 31 of annular piston 9 increasingly compresses the movable body 39, this movable body 39 being formed by the stack of linings described in FIG. 2, which compresses, and by the portion 1 of segment 15 which is increasingly clamped between this stack of linings and the lower part of the body 38 of radial brake 7 on which the movable body 39 is pressing more and more, the hydraulic pressure or thrust exerted by annular piston 9 on the movable body 39 being symbolized by arrow 62,
   such that the movable body 39 is prevented from rotating around the body 38 traversing it.

Of course, the invention is not limited to the examples and to the embodiment which are described and represented, but is capable of numerous variants accessible to those skilled in the art.

1. Articulated support arm for an elongated flexible medical instrument, comprising:
a segment rotating about an axis of rotation,
a radial brake for preventing rotation of the segment about the axis of rotation, comprising:
a supply of hydraulic fluid,
a piston whose stroke along the direction of the axis of rotation, due to the pressure of the hydraulic fluid, prevents rotation of the segment about the axis of rotation,
the piston being an annular piston having a hollow center about the axis of rotation,
the supply of hydraulic fluid passing through said hollow center.

2. The articulated arm according to claim 1, wherein said stroke of said piston prevents the rotation of said segment about the axis of rotation but does not lock said segment.

3. The articulated arm according to claim 1, further comprising:
an axial pivot which physically defines the axis of rotation and around which the segment rotates,
the axial pivot passing through said hollow center,
a central channel for the supply of hydraulic fluid, passing inside the axial pivot and through said hollow center.

4. The articulated arm according to claim 3, wherein the central channel for the supply of hydraulic fluid extends in the direction of the axis of rotation.

5. The articulated arm according to claim 4, wherein the central channel for the supply of hydraulic fluid extends along the axis of rotation.

6. The articulated arm according to claim 3, further comprising a radial channel for the supply of hydraulic fluid, extending radially to the axis of rotation and connecting the central channel to the annular piston.

7. The articulated arm according to claim 3, wherein at least one of the axial ends of the axial pivot comprises a hydraulic connection including a rotating gasket.

8. The articulated arm according to claim 3, wherein the segment rotates about the axial pivot by means of a needle bearing or a plurality of needle bearings which is/are radially positioned between the axial pivot and the segment.

9. The articulated arm according to claim 3, further comprising two of said rotation-preventing radial brakes with annular piston, located respectively at two axial ends of the axial pivot.

10. The articulated arm according to claim 3, wherein the segment has two ends which respectively rotate about two of said axial pivots whose axes of rotation have the same direction.

11. The articulated arm according to claim 10, wherein the segment is rectilinear and inclined relative to the direction of the axes of rotation of the two axial pivots.

12. The articulated arm according to claim 10, wherein the two axial pivots are interconnected by a hydraulic fluid supply line.

13. The articulated arm according to claim 12, wherein the hydraulic fluid supply line is rectilinear and orthogonal to the direction of the axes of rotation of the two axial pivots.

14. The articulated arm according to claim 1, wherein:
the radial brake comprises:
a lining attached to the annular piston,
a lining attached to the segment,
an unattached lining which is not attached to either the annular piston or the segment,
the material of the unattached lining being less hard than the material or materials of the attached linings,
the attached linings and the unattached lining being arranged relative to one another such that, when the hydraulic fluid pushes the annular piston, the unattached lining is clamped between the two attached linings so as to prevent rotation of the segment about the axis of rotation.

15. The articulated arm according to claim 14, wherein:
the lining attached to the annular piston is made of steel,
the lining attached to the segment is made of steel,
the unattached lining is made of plastic.

16. The articulated arm according to claim 1, further comprising:
at least three segments articulated to rotate with respect to each other about a same rotation direction, which include:
a proximal segment, located closest to the operating table when the articulated arm is fixed to this operating table,
a distal segment, carrying the elongated flexible medical instrument,
an intermediate segment, located between the proximal segment and the distal segment.

17. The articulated arm according to claim claim 1, further comprising:
at least four segments articulated to rotate with respect to each other about a same rotation direction, which include:
a proximal segment, located closest to the operating table when the articulated arm is fixed to this operating table,
a distal segment, carrying the elongated flexible medical instrument,
at least two intermediate segments, located between the proximal segment and the distal segment.

18. The articulated arm according to claim 1, wherein the elongated flexible medical instrument comprises a catheter and/or a catheter guide and/or a guide catheter.

19. Method for preventing the mobility of an articulated support arm for an elongated flexible medical instrument, comprising a segment that rotates about an axis of rotation and a radial brake for preventing rotation of the segment about the axis of rotation, comprising:
a supplying of hydraulic fluid through a hollow center of an annular piston of the radial brake,
a stroke of said piston along the direction of the axis of rotation, due to the pressure of said hydraulic fluid, so as to prevent rotation of the segment about the axis of rotation.

20. The method of claim 14, wherein the unattached lining is clamped between the two attached linings so as to prevent rotation of the segment about the axis of rotation without locking the segment.
